(19) United States
(12) Patent Application Publication
(10) Pub. No.: US 2010/0056857 A1
(43) Pub. Date: Mar. 4, 2010

(54) URETHRAL SLINGS, AND METHODS FOR THE IMPLANTATION AND ADJUSTMENT THEREOF

(75) Inventor: Michael Nordmeyer, Pittstown, NJ (US)

Correspondence Address: FOX ROTHSCHILD LLP, 997 Lenox Drive, Bldg. #3, Lawrenceville, NJ, 08648, US

(73) Assignee: BIHLER OF AMERICA, INC., Phillipsburg, NJ (US)

(21) Appl. No.: 12/550,545

(22) Filed: Aug. 31, 2009

Related U.S. Application Data
(60) Provisional application No. 61/093,308, filed on Aug. 30, 2008.

(51) Int. Cl. A61F 2/04

(52) U.S. Cl. 600/30

(57) ABSTRACT

The present invention relates to a urethral sling for treating incontinence, including methods of manufacturing the sling; methods of implanting the sling and methods of adjusting the sling both during and after installation. More specifically, the sling includes a collapsible tubular mesh of fibers designed to support the urethra and allow for tissue growth while easily conforming to the patient. The sling is pre-sized to eliminate the need for cutting to width during manufacture or installation and, thereby, eliminating the risk of creating rough or sharp edges. It is further equipped with one or more installation features for facilitating installation and securing the sling within the patient.

### CROSS-REFERENCE TO RELATED APPLICATIONS

This application claims priority to U.S. Provisional Patent Application Ser. No. 61/093,308 filed Aug. 30, 2008, the disclosure of which is incorporated by reference herein.

### FIELD OF THE INVENTION

The present invention relates to a urethral mesh sling for treating incontinence, including methods of manufacturing the sling; methods of implanting the sling and methods of adjusting the sling both during and post installation.

### BACKGROUND OF THE INVENTION

It is estimated that over 19 million North American adults have urinary incontinence. The condition can range in severity from partial to complete loss of bladder control with varying degrees of urine loss. Generally speaking, incontinence is not considered a disease, but rather a symptom or side effect of some other medical condition(s). Some conditions known to cause male urinary incontinence include prostate surgery, prostatectomy, head and spinal cord injury, infection, certain toxins (e.g. alcohol, medications, etc.) and certain diseases such as cancer, Parkinson's disease, and multiple sclerosis. Male incontinence is also associated with the aging process. In short, male incontinence can be associated with a myriad of factors.

Women account for an estimate 11 million incontinence cases, which in many instances is caused from stress urinary incontinence (SUI). SUI may be caused by a functional defect of the tissue or ligaments connecting the vaginal wall with the pelvic muscles and pubic bone. Common causes include repetitive straining of the pelvic muscles, childbirth, loss of pelvic muscle tone, and estrogen loss. Such a defect results in an improperly functioning urethra, and affected women involuntarily lose urine during normal daily activities and movements, such as laughing, coughing, sneezing and regular exercise. Unlike other types of incontinence, SUI is not a problem of the bladder.

Normally, the urethra, when properly supported by strong pelvic floor muscles and healthy connective tissue, maintains a tight seal to prevent involuntary loss of urine. When a person suffers from the most common form of SUI, however, weakened muscle and pelvic tissues are unable to adequately support the urethra in its correct position. As a result, during normal movements when pressure is exerted on the bladder from the diaphragm, the urethra cannot retain its seal and permits urine to escape. Because SUI is both embarrassing and unpredictable, many people with SUI often avoid an active lifestyle, shying away from social situations.

One general solution for both male and female incontinence, particularly SUI, is the use of implants or mesh slings to support the urethra and, in some cases, the bladder. Such slings are typically made in flat sheets or strips, as illustrated in U.S. Pat. Nos. 7,556,598; 7,303,525; 7,070,558; and 6,911,002. The slings are typically made at a uniform length and cut to a narrower width or size to be suitable for implanting into the patient. The problem associated with cutting the woven mesh is that it is prone to having rough or sharp edges. Inserting the sling then becomes difficult because the edges can snag the muscular tissue or fascia. This hinders placement within the patient and often requires additional elements, such as plastic sleeves, to facilitate installation and minimize tissue damage. Additionally, post-insertion, patients often complain that the rough edges of the sling cause discomfort from rubbing against the adjacent tissue. Finally, the flat sheet construction of the sling often makes adjusting the sling, or readjusting post-operatively, difficult.

Accordingly a urethral sling is desirable that treats SUI or similar incontinence and that may be easily inserted into the patient without creating rough or sharp edges or other discomfort to the patient. Moreover, a urethral sling is desirable that may be easily adjusted post-installation, or post-operatively, while causing minimal discomfort to the patient. The instant invention addresses the foregoing needs along with methods of inserting and adjusting the sleeve, as well as manufacturing it.

### SUMMARY OF THE INVENTION

The present invention relates to a urethral sling for treating incontinence, including methods of manufacturing the sling; methods of implanting the sling and methods of adjusting the sling both during and after installation. More specifically, the sling includes a collapsible tubular mesh of fibers designed to support the urethra and allow for tissue growth while easily conforming to the patient. The sling is pre-sized to eliminate the need for cutting to width during manufacture or installation and, thereby, eliminating the risk of creating rough or sharp edges. It is further optionally equipped with one or more installation features for facilitating installation and securing the sling within the patient.

In one embodiment, the sling includes a mesh of individual small gage fibers or bundles of fibers woven to form a tubular urethral sling. The fibers are formed from a biocompatible, bioabsorbable, and/or thermoplastic material such as, but not limited to, polypropylene, polydioxanone, or any one or combination of polymers discussed herein. In certain embodiments, the fibers are woven along their longitudinal axis to form a flat sheet and provide for one or more ridges or installing devices (e.g. openings, saddles, tips, etc.) described herein. The width of the mesh is then rolled into a final tubular structure and the two opposing sides of the mesh sealed together using one or more mechanisms discussed herein.

Because of the flexible nature of the fibers, the composition of the tubular mesh is adapted to collapse into a flattened state once implanted in the patient, particularly in view of the pressure exerted on the mesh by the surrounding muscle, tissue, or fascia. The diameter of the mesh, accordingly, can be chosen so that the width of the sling when flattened is sufficient to provide adequate support to the urethra, and to facilitate adequate tissue growth around the mesh.

One or more tips are optionally positioned at either or both opposing ends of the mesh and includes a body, which is coupled to the mesh, and an adjoining end portion, which protrudes from the mesh. The body can be formed as a hollow cylinder, and the end portion of each tip as a hollow or semi-hollow conical shape that terminates in a point. This latter feature facilitates tip penetration into muscle, tissue, and fascia during implantation and securing the mesh within the patient after installation. The composition of the tip may be the same or different from that of the sling fibers, as discussed further herein, but is generally more rigid so as to facilitate installation.

An opening can be formed at or near the lengthwise center of the mesh, to facilitate the insertion of a rigid or semi-rigid tube-shaped applicator. The opening could be woven or knitted in to the tubular mesh as part of the weaving process, discussed herein, to improve the strength of the fibers at this position and eliminate the risk of cut edges. In alternative embodiments, the opening is not limited to the lengthwise center of the mesh and also can be formed at locations other than the lengthwise center of the mesh in alternative embodiments.

The sling may also include a pad or saddle mounted on or woven into the mesh for contacting the urethra when the sling is implanted in the patient. The saddle can be formed from a rigid, semi-rigid, or flexible material that will not undergo a reduction in width as the sling is tensioned during implantation. To this end, the saddle facilitates maintaining a flat shape of the sling after installation and does not cause discomfort to the patient. In non-limiting embodiments, the saddle includes a conforming biocompatible fleece or pad made from absorbable or non-absorbable material.

The sling of the instant invention can be implanted surgically inside a patient experiencing urinary incontinence in a manner that supports the patient's urethra so as to relieve the incontinence. In one embodiment, an applicator may include a rigid rod-like apparatus, which is sized to fit within the opening of the mesh and extend into the end of the mesh and tip, where applicable. Once fully inserted into the sling, the applicator and sling is inserted into the patient via an incision and pushed to the desired location within the patient.

The shape of the tip can retain the sling in position within the patient until in-growth of tissue takes place. Alternatively, the sling may be provided without the tip element and is secured within the patient using tension from the muscle, tissue or fascia to grip the sling in place along its length. In either case, the applicator is then withdrawn from the sling, leaving the sling in place inside the patient. As it is withdrawn, the mesh is compressed and collapsed into a flat configuration due to the pressure exerted by the surrounding muscle, tissue, or fascia. These steps are performed on both ends of the sling until the sling is secured therewithin. In certain embodiments, the end portions of the mesh are installed such that the saddle contacts the area between the sling and the urethra and maintains the flattened configuration on and around the urethra.

Post-installation, or on a post-operative basis, a fit check of the sling can then be performed to determine if there is adequate tension of the sling to support the urethra, or if the sling requires readjustment. If so, the applicator can be reinserted into the opening of the tubular mesh and guided to one or both ends of the mesh.

In an alternative embodiment, the tension in the sling may also be adjusted without moving the tips; but rather, shrinking or stretching the mesh using an energy source, such as heat, light, etc. While not limited thereto, in one embodiment, shrinking or stretching the sling is facilitated using a gripping apparatus, which includes at least one or multiple grips to spread and contract the mesh sling. Each grip includes two handles for grasping the mesh on either or both sides of the mesh. Each grip may be moved toward and away from each other, and locked in position, as provided herein.

A energy source and, optionally, a protective shield also can be moved into proximity or contained within the handle or body of the gripping apparatus and is adapted, in accordance with the teachings herein, to induce shrinkage or stretching in the mesh. To this end, the energy source is used to adjust the fit of the urethral sling in the patient by either shrinking or stretching the mesh, in accordance with the methods provided herein. The energy source may be comprised of, but is not limited to, a heat source, or any temperature source, or a light source (e.g. IR light, UV light, etc.).

In alternative embodiments, however, shrinking or stretching the sling may be accomplished without use of a gripping apparatus. Rather, the length of the sling may be reduced or stretched by simply applying the energy source to a localized area of the sling. The amount of stretching or shrinkage may be a function of the intensity of the energy source combined with known properties of the fibers or bundles of fibers used within the woven mesh.

### BRIEF DESCRIPTION OF THE DRAWINGS

The foregoing summary, as well as, the following detailed description of preferred embodiments, are better understood when read in conjunction with the appended drawings. The drawings are presented for illustrative purposes only, and the scope of the appended claims is not limited to the specific embodiments shown in the drawings. In the drawings:

FIG. 1 is a perspective view of an embodiment of a urethral sling, and a urethra being supported by the urethral sling.

FIG. 2 is a perspective view of a tubular mesh and a saddle of the urethral sling depicted in FIG. 1, which may be internal or external to the tubular mesh.

FIG. 3 is a side view of the tubular mesh and saddle shown in FIG. 2.

FIG. 4 is a front view of the urethral sling shown in FIGS. 1-3.

FIG. 5 is a top view of the urethral sling shown in FIGS. 1-4.

FIG. 6 is a magnified view of the area designated “FIG. 6” in FIG. 4, showing a tip of the urethral sling in cross section.

FIG. 7 is a front view of the urethral sling shown in FIGS. 1-6, and an applicator used to implant the urethral sling.

FIG. 8 is a magnified view of a tip of the urethral sling and depicts the applicator shown in FIG. 7 engaging the tip.

FIGS. 9A-9C are magnified views of FIG. 7, showing the tip of the urethral sling in cross section, and depicting the tip implanted in a patient and the applicator being withdrawn following implantation.

FIG. 10 depicts a mesh of the urethral sling shown in FIGS. 1-9C being stretched or contracted by a gripping device, including an energy source to shrink or lengthen the urethral sling.

FIG. 11 depicts the mesh and gripper as shown in FIG. 10, taken from a side viewpoint.

FIG. 12 is a magnified view of the area within the energy source and protective shield in FIG. 11.

FIG. 13 depicts the mesh and gripper as shown in FIGS. 10-12, taken from a viewpoint rotated approximately ninety degrees for the viewpoints of FIGS. 10-12.

FIG. 14 illustrates one embodiment for manufacturing the tubular mesh of the instant invention without a tip.

FIG. 15 illustrates another embodiment for manufacturing the tubular mesh of the instant invention with one or more tip elements.

FIG. 16 illustrates another embodiment for manufacturing the tubular mesh of the instant invention with a saddle element.

FIG. 17 is a front view of an alternative embodiment of the urethral sling illustrating a saddle that is woven into the mesh.

FIG. 18 is a top view of the urethral sling shown in FIG. 17.

FIG. 19 is a front view of the urethral sling shown in FIGS. 17, and an applicator used to implant the urethral sling.

### DETAILED DESCRIPTION

The present invention relates to a urethral sling for treating incontinence, including methods of manufacturing the sling; methods of implanting the sling and methods of adjusting the sling both during and after installation. More specifically, in certain embodiments, the sling includes a collapsible tubular mesh of one or more small gage fibers or bundles of fibers designed to support the urethra and allow for tissue growth while easily conforming to the patient. The sling is pre-sized, using manufacturing techniques discussed herein, to eliminate the risk of creating rough or sharp edges during manufacture or otherwise during installation. It is optionally equipped with one or more rigid or semi-rigid tips, ribs, saddle and/or an access port any of which facilitate insertion and securing the sling within the patient, as also discussed herein.

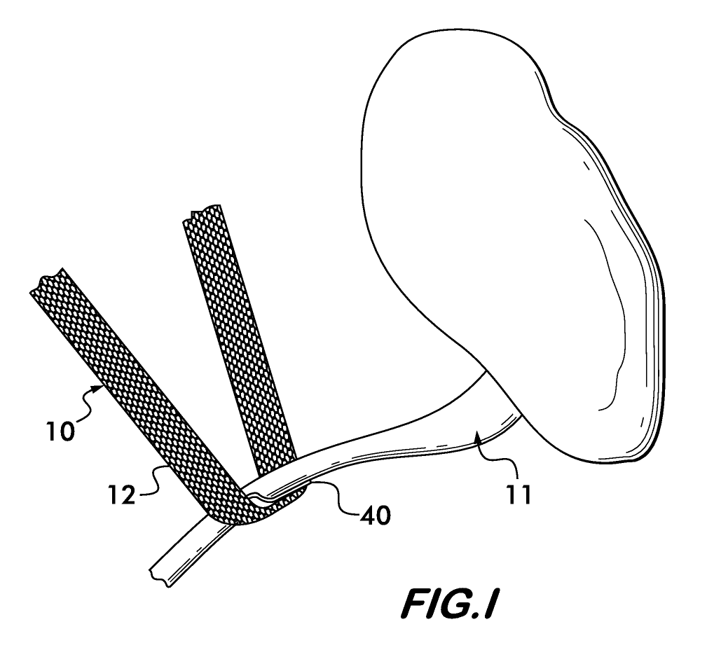

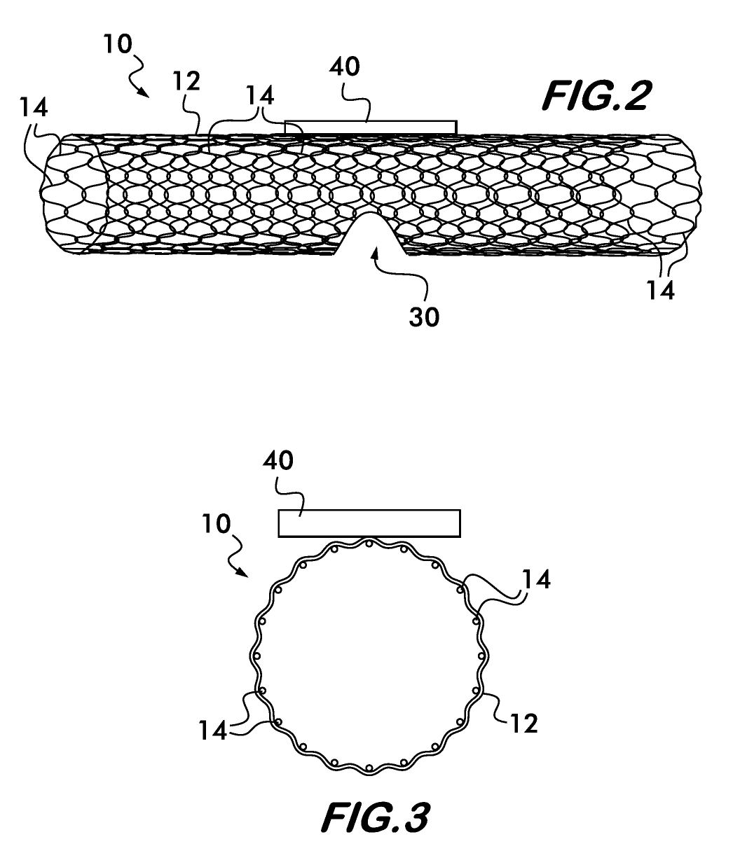

Referring to FIGS. 1 and 2, one embodiment of a urethral sling 10 is illustrated. The sling 10 includes a mesh 12 of individual fibers or bundles of fibers 14, which are woven in a cross-sectional pattern, such as but not limited to that shown in FIG. 2. As used herein, the terms fibers and bundles of fibers are interchangeable such that an reference to the term “fiber” includes a monofilament fiber as well as a bundle of fibers or any other configuration of fibers that is known in the art. To this end, the term fiber can include either a bundle of numerous much smaller fibers (e.g. a size range of 5 denier or less) or a single mono filament fiber. In one embodiment, the fibers 14 are formed from either or a combination of polypropylene or polydioxanone. The instant invention, however, is not so limited and the fibers 14 also may be formed from other similar thermoplastic materials or other biocompatible, bioabsorbable and/or non-absorbable materials that are known in the art, including, but not limited to, nylon, polyethylene, polyester, fluoropolymers, and/or copolymers thereof. In even further alternatives, the instant invention is not necessarily limiting to a mesh structure, and may be comprised of any other material and any other configuration so as to be in a tubular shape and in accordance with the methods and advantages discussed herein.

In embodiments with a woven mesh, the fibers 14 may be woven or otherwise constructed to achieve the objectives and advantages discussed herein. As illustrated in FIG. 2, in one embodiment, the fibers are woven along their longitudinal axis forming a mesh 12 having a substantially circular cross section. The fiber composition of the mesh 12 may be specifically knitted to achieve various stretch characteristics of the sling and account for one or more of the installing devices described herein.

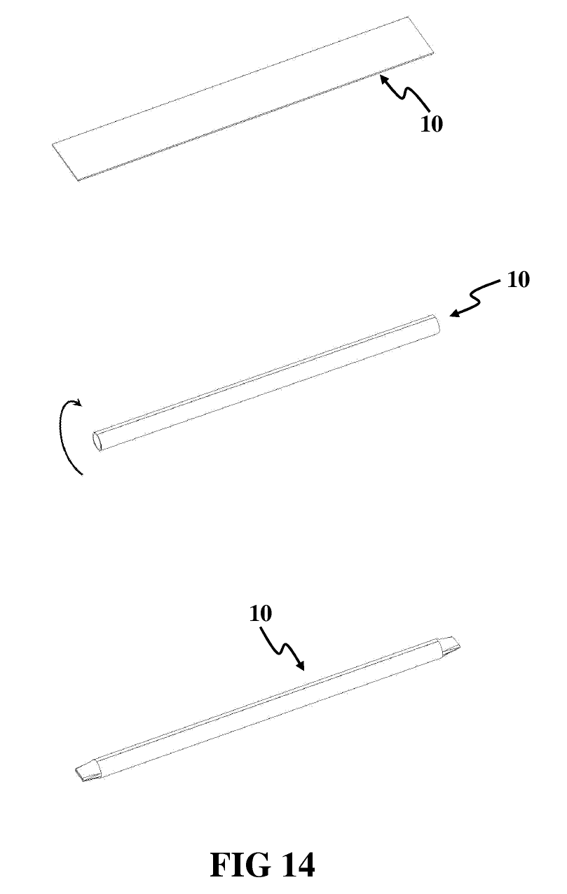

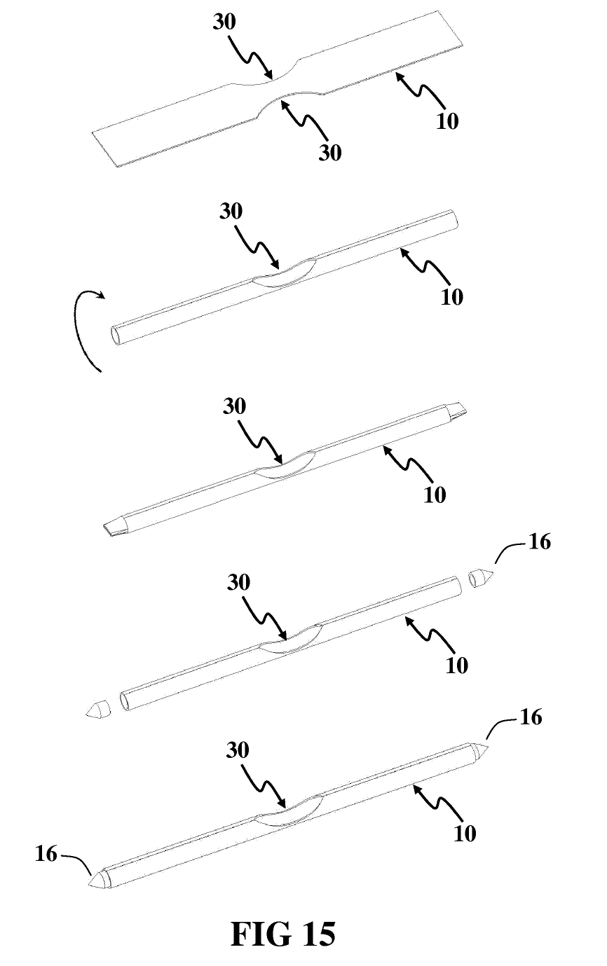

Referring to FIGS. 14-16, in one embodiment, the fibers 14 are continuously woven to a flat length of mesh or a single layer. The mesh 12 for a particular sling 10 can be woven to a pre-determined length and to approximately twice the width for the application in which that particular sling 10 will be used. Alternatively, the mesh may be woven into a flat sheet, and a pre-determined length and twice the width cut from the sheet. In either case, the width of the mesh 12 is then rolled about its longitudinal axis into a final tubular structure such that the two opposing sides of the mesh interface and are sealed together. While non-limiting to the invention, the two sides may be sealed by conventional mechanisms known in the art such as, but not limited to, suturing, ultrasonic welding, or heat sealing. Alternatively, they may be sealed using any biocompatible adhesive or any means otherwise known in the art for bonding fibers. The length of the sling may be between approximately 6-18 cm. In certain embodiments, the length is between 8-14 cm. In even further embodiments, the length is between 10-12 cm. Such size ranges are not necessarily limiting to the instant invention, however, and any sized sling may be used, depending on where the sling is being anchored within the patient.

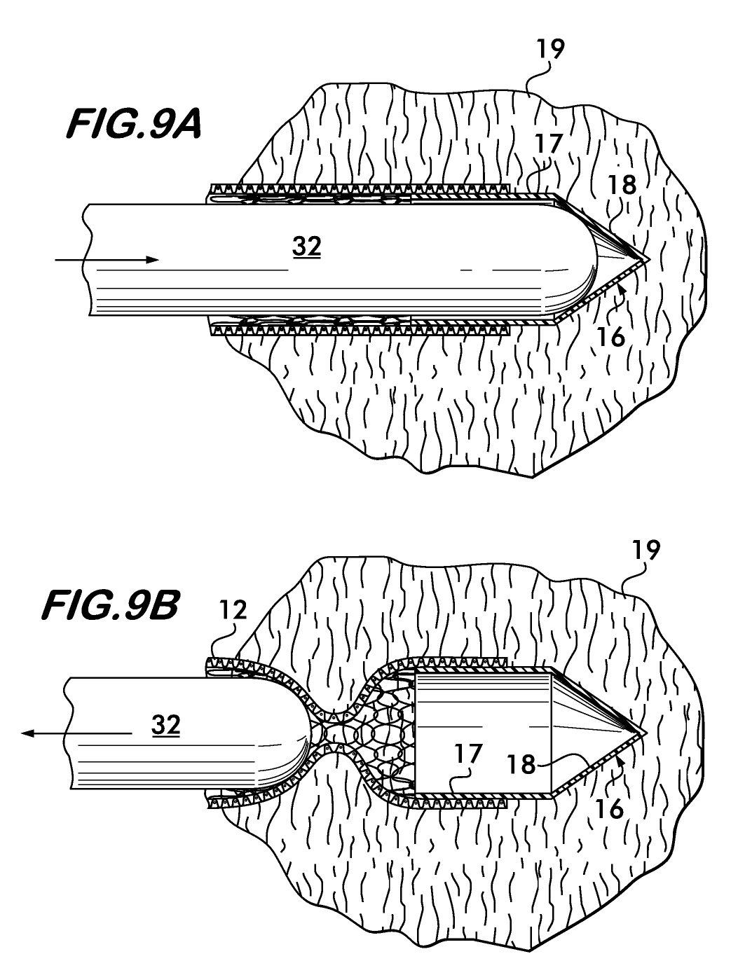

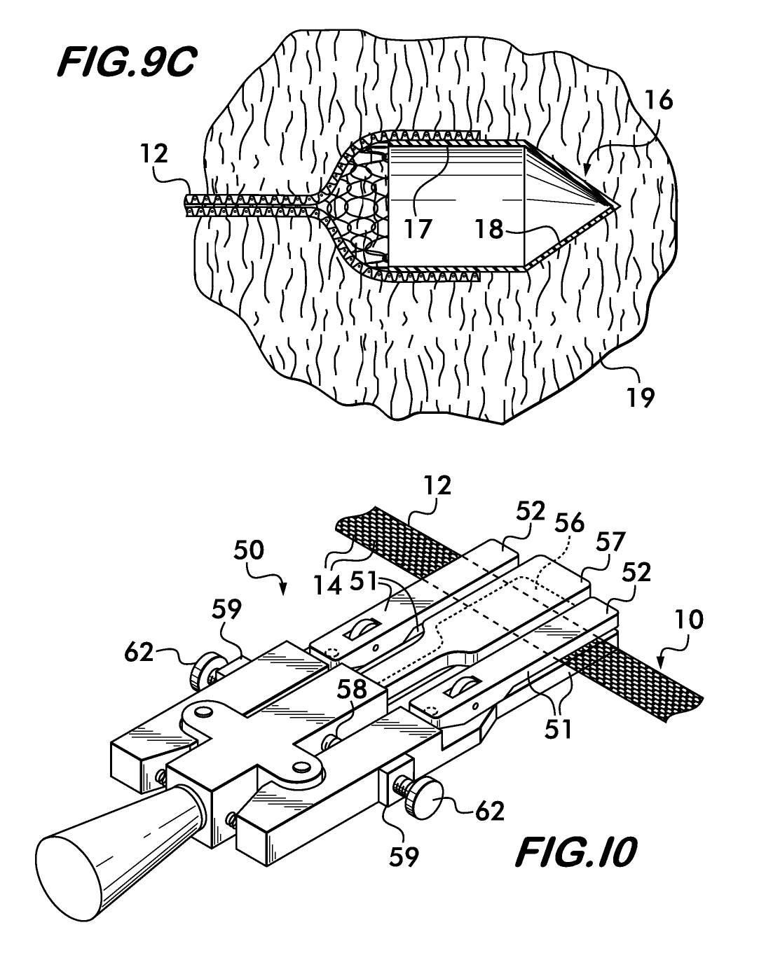

Because of the flexible nature of the fibers 14, the composition of the tubular mesh 12 is adapted to collapse into a flattened state and remain relatively flat once implanted in the patient, as shown in FIGS. 9B and C, due to the pressure exerted on the mesh 12 by the surrounding muscle, tissue, or fascia 19. The diameter of the mesh 12, accordingly, can be chosen so that the width of the sling 10 when flattened is sufficient to provide adequate support to the urethra 11, and to facilitate adequate tissue growth around the mesh 12. Adequate tissue growth around the mesh 12 is needed to provide satisfactory retention of the sling 10.

The tubular construction of the mesh 12 provides the sling 10 with more cross-sectional area, and greater strength in comparison to a conventional flat-mesh sling of comparable width. Thus, the sling 10 can be woven from fibers 14 having a smaller gage than the fibers of a conventional flat-mesh sling with a comparable, or lower load-bearing capacity. The lower gage fibers 14 can help the mesh better conform to the patient, and can make the sling 10 less intrusive, which in turn can reduce the levels of post-operative pain and discomfort. In one non-limiting embodiment small gage fibers may include fibers having a diameter between 0.02 mm and 0.8 mm.

Although not illustrated in the accompanying figures, the instant sling may also include one or more ribs or features to the inside or outside of the tubular mesh that promote it maintaining the sling width when under tension. The ribs may be formed from weaving another fiber or material into the tubular mesh at an angle or perpendicularly to the length of the tube. The fiber may be comprised of the same fiber(s) as the mesh 12 or may be comprised of one or more alternative fibers otherwise contemplated herein.

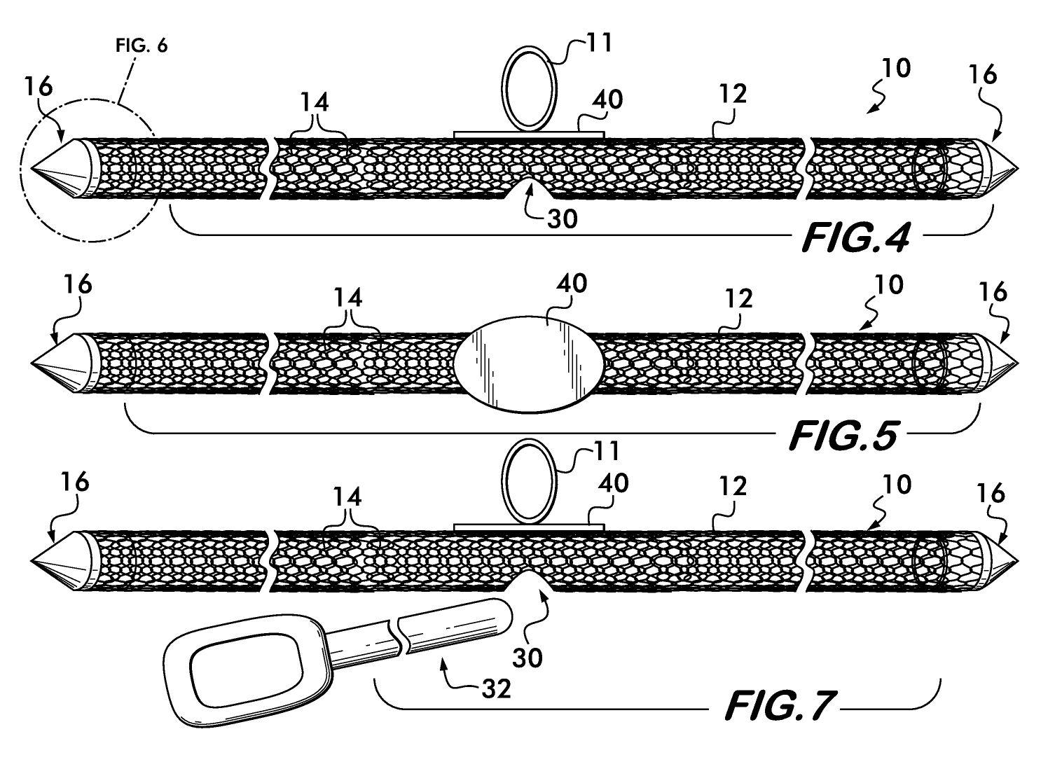

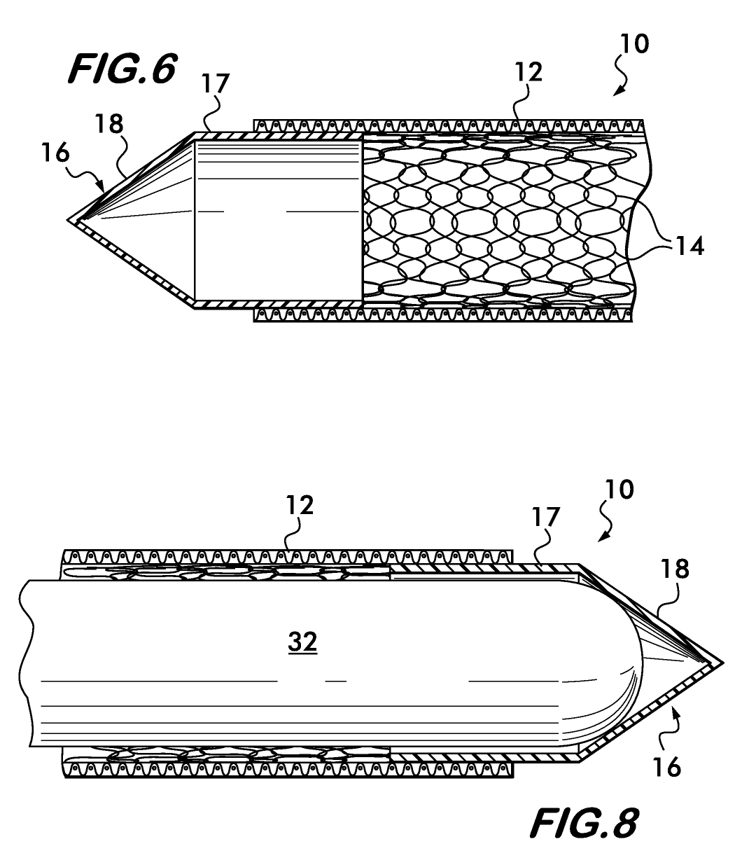

Referring to FIGS. 4-6, a tip 16 may be optionally positioned at either or both opposing ends of the mesh 12 and adapted to facilitate guiding and securing the sling within the patient. Each tip 16 includes a body 17 and an adjoining end portion 18, as more clearly illustrated in FIG. 6. The body 17 can be formed as a hollow cylinder, and the end portion 18 of each tip 16 as a hollow or semi-hollow conical shape that terminates in a point. This latter feature facilitates tip 16 penetration into muscle, tissue, and fascia 19 during implantation, as shown in FIGS. 9A-9C. The end portion 18 is not necessarily limited to this configuration and can be entirely solid. In even further alternative, the tip 16 is not necessarily limited to such shape, however, and may include any shape to assist in the installation and retention of the sling.

In certain embodiments, the tip(s) 16 is formed from a rigid, semi-rigid, or flexible material that is adapted to facilitate installation and permanent implantation. The composition of the tip may be the same or different from that of the sling fibers. To this end, in one embodiment, the tip is formed from polypropylene or polydioxanone. The instant invention, however, is not so limited and the tip also may formed from other similar thermoplastic materials or other biocompatible, bioabsorbable or non-absorbable materials that are known in the art. Such materials include, but are not limited to, nylon, polyethylene, polyester, fluoropolymers, and/or copolymers thereof.

The tip 16 may be secured to the ends of the sling using standard means known in the art. To this end, the end of the mesh 12 can be bonded to an outer or inner surface of the body 17 using a biocompatible adhesive or by otherwise, fusing, welding or melting the mesh 12 to the tip 16. As illustrated in FIG. 6, in certain non-limiting embodiments, the mesh is secured to the body of the tip using one or a combination of the methods above such that at least a portion of the adjoining end 18 protrudes from the end portion of the mesh sling 10. This facilitates installing the sling and also provides a mechanism for securing the sling to the tissue of the patients.

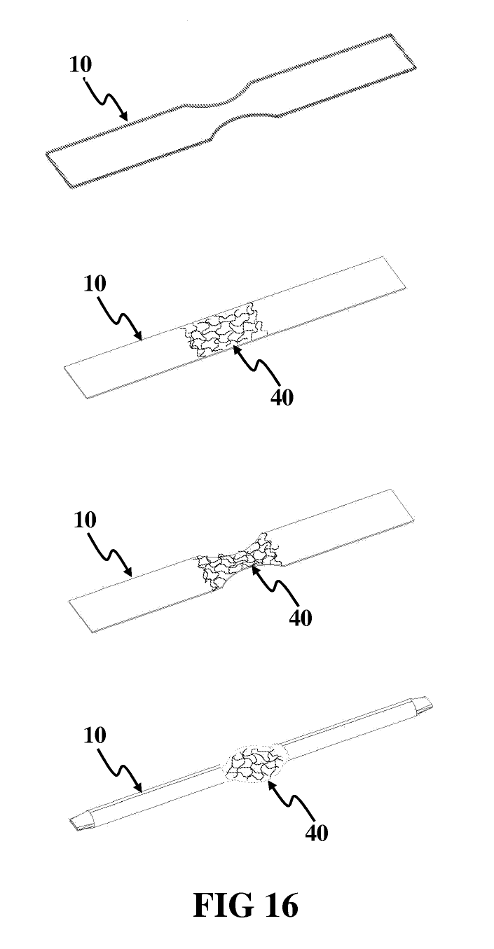

In alternative embodiments, as illustrated in FIGS. 14 and 16, the instant sling does not require a tip 16. In such embodiments, the end portions of the tubular mesh sling are sealed together, such as by knitting, stitching, sealing, bonding, fusing, welding or gluing. In such embodiments, the tubular sling 10 is secured to the patient, by relying on the muscle, tissue or fascia to grip or frictionally secure the sling in place along its length. Such a position becomes more permanent once the tissue growth in and around the surgical site of insertion re-grows.

One or more additional features of the sling may be included in any of the above embodiments to facilitate positioning the sling within the patient. For example, an opening 30 can be formed at or near the lengthwise center of the mesh 12, to facilitate the insertion of a rigid or semi-rigid applicator 32 as shown in FIGS. 7-9A and 15. The opening for the inserter could be woven or knitted in to the tubular mesh as part of the weaving process, discussed herein, to improve the strength of the fibers at this position and eliminate the risk of cut edges. The instant opening is not limited to the lengthwise center of the mesh 12 and also can be formed at locations other than the lengthwise center of the mesh 12 in alternative embodiments. The applicator 32 may be comprised a stainless steel wire that is approximately 2 mm-4 mm in diameter and a length that is approximately 6 cm-20 cm. The instant invention, however, is not limited by such dimensions and similar applicators otherwise known in the art may be adapted for use in the methodology discussed herein.

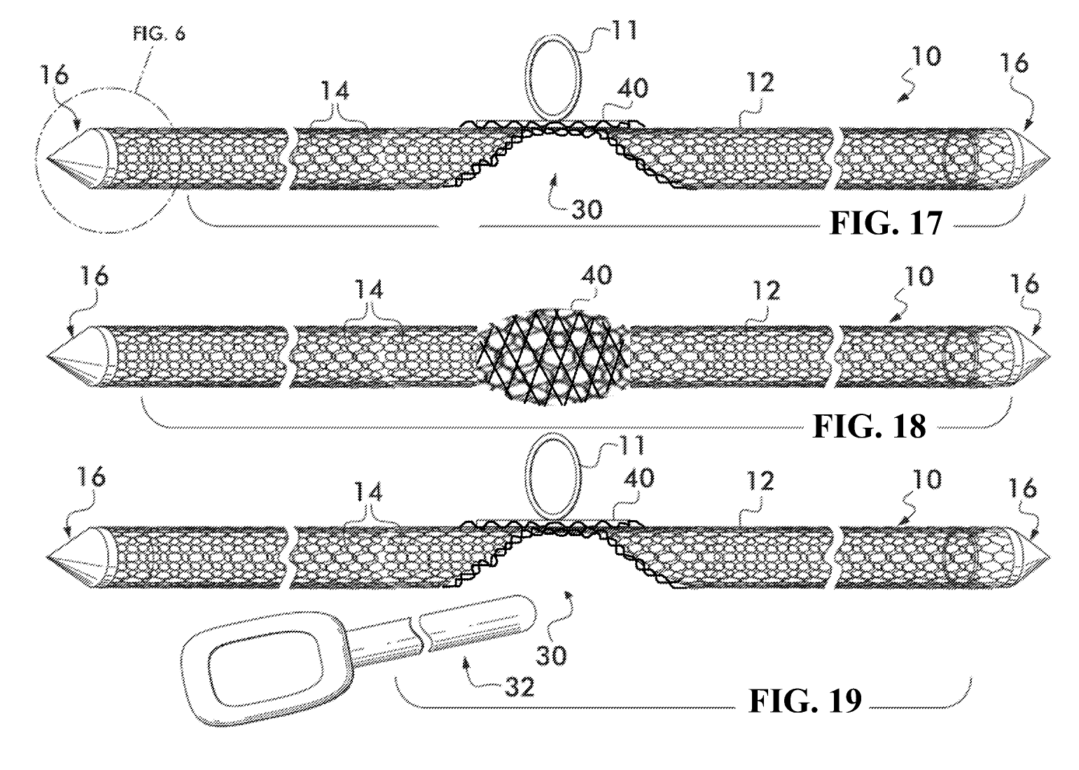

In further embodiments, the sling 10 also includes a pad or saddle 40, as shown in FIGS. 1-5 and 17-19. The saddle 40 can be mounted on (as in FIGS. 1-5) or woven within (as in FIGS. 17-19) the mesh 12 so that it may be positioned to contact the urethra 11 when the sling 10 is implanted in the patient, as shown in FIG. 4. The saddle 40 can be formed from a rigid, semi-rigid, or flexible material that will not undergo a reduction in width as the sling 10 is tensioned during implantation, and that will not cause discomfort to the patient. In non-limiting embodiments, the saddle includes a conforming biocompatible fleece or pad made from absorbable or non-absorbable material. The pad may be approximately 5 mm -30 mm in length and approximately 1 mm -10 mm in width. The typical length of support needed is 10 mm-20 mm.

The saddle 40 can be woven into the sling 10 or alternatively attached to the mesh 12 by a suitable means such as adhesive, welding, bonding, fusing or stitching. The saddle 40 can provide additional contact area between the sling 10 and the urethra 11, thus reducing the potential for the urethra 11 to be worn or eroded by the mesh 12. This feature can be particularly useful, for example, to prevent the mesh 12 from stretching, bunching, or curling when put under tension, as is normally seen done in an SUI procedure. Because such stretching, bunching or curling can reduce the effective contact area of the mesh 12, which in turn can increase the potential for wear and erosion of the urethra 11, the saddle 40, optionally in combination with the fibers, acts to aid in holding the center area of the sling 10 in a relatively flat configuration.

In each of the foregoing embodiments of the instant invention, the mesh 12 of the sling 10 is tubular and pre-formed to a known width or shape; it does not need to be cut to size in the width-wise direction during manufacture or during the procedure in order to achieve the correct size. Thus, the sling 10 does not have any exposed edges or loose ends along its width. The ends of the mesh 12 are either attached to the tips 16 or otherwise sealed such that there are no loose exposed ends along the length. The lack of exposed edges or loose ends facilitates inserting the sling 10 through muscle, tissue, or fascia 19 when implanting the sling 10 in the patient. This, in turn, enhances accuracy in positioning the sling 10, results in reduced pain or discomfort to the patient, and eliminates the need for a plastic or film sheath over the sling 10 during implantation.

Based on the foregoing, the sling 10 of the instant invention can be implanted surgically inside a patient experiencing urinary incontinence in a manner that supports the patient's urethra 11 so as to relieve the incontinence. Referring to FIG. 7, an applicator may include a rigid rod-like apparatus, which may be rounded at one end and contains a handle at the other. As illustrated in FIGS. 7, 8, and 9A, the applicator 32 could have a rounded end and can be sized to fit within the opening 30 of the mesh 12. The tubular configuration of the sling 10 can facilitate the insertion of an applicator or inserter therethrough to and into the body 17 of tip 16. The applicator used to install the instant invention is not limited to a rounded tip, however, and could also include one or more features (e.g. ridges, ribs, teeth, or the like) to facilitate locking the applicator into the tips 16 or the end of the mesh sling.

As illustrated in FIGS. 8 and 9A, the applicator 32, once fully inserted into the sling 10, can be inserted into the patient along with the sling 10 via an incision made in the patient. The applicator 32 can then be pushed into the patient to position the sling 10 at a desired location, as shown in FIG. 9A, such that the muscle, tissue and/or fascia is able to frictionally secure or grip the sling (and tip if used) within the patient. Such methods of installation are consistent with those known and performed within the art. In one non-limiting embodiment, the targeted location may be the dense connective, muscular, or membrane tissue of the pelvic region.

The tips 16, where present, can assist in retaining the sling 10 in position within the patient until in-growth of tissue takes place. The tips 16 of alternative embodiments can be equipped with features that cause the tips 16 to grasp the surrounding muscle or fascia to further secure the tips 16 in place after insertion. For example, ridges or teeth can be formed along the outer perimeter of the body 17 of each tip 16 to increase the retentive force exerted by the surrounding muscle or fascia on the tip 16. In alternative embodiments of the invention without one or more tips 16, the sling may be directly secured to the target location, wherein the target location may the dense connective, muscular, or membrane tissue of the pelvic region.

In either case, once one end of the sling 10 is secured to the tissue, the applicator 32 is withdrawn from the sling 10. Referring to FIGS. 9A-9C, as the applicator 32 is withdrawn back through the opening 30 and the mesh 12 is compressed and collapsed due to the pressure exerted by the surrounding muscle, tissue, or fascia 19. The tubular configuration of the mesh 12 makes the instant invention particularly advantageous to this effect because the mesh 12 is well-suited to conform to the surrounding muscle, tissue, or fascia 19 due to the pressure exerted thereby, which in turn can minimize the potential for patient discomfort. In embodiments of the invention containing rigid tips 16, these elements are of sufficient rigidity such that they do not collapse, as shown in FIG. 9C.

These steps are performed on both ends of the sling such that both end portions are secured within the patent as illustrated in FIG. 1. In certain embodiments, the end portions of the mesh 12 are installed such that the saddle 40 contacts the area between the sling 10 and the urethra 11, as illustrated in FIG. 1. The additional contact area can reduce the potential for the urethra 11 to be worn or eroded by the mesh 12. This feature can be particularly useful when, for example, the mesh 12 stretches or curls when put under tension, as is normally done in an SUI procedure. Such stretching or curling can reduce the effective contact area of the mesh 12, which in turn can increase the potential for wear and erosion of the urethra 11.

Post-installation, a fit check of the sling 10, free of the influence of the applicator 32, can then be performed to determine if the position of the sling 10 requires readjustment. If so, the applicator 32 can be reinserted into the opening in the tubular mesh 12. The tubular configuration of the mesh 12 can facilitate guiding the applicator 32 toward one of the tips 16 of the sling 10 at one or both ends. The tip 16 can be pushed using the applicator 32, if a tighter fit that provides more urethral support is required.

The tip 16 and the end of the applicator 32 can be equipped with features (not shown) such as latches, projections and slots, complementary threads, etc., that permit the end of the applicator 32 to be secured to the tip 16. Securing the applicator 32 to the tip 16 permits the tip 16 to be pulled in a direction away from the direction of insertion, if the tension in the sling 10 needs to be lessened to provide a looser fit around the urethra 11.

The tension in the sling 10 can also be readjusted on a post-operative basis, if necessary, days, weeks, months, or years after the implantation procedure. In particular, the incision can be opened in the patient, and the applicator 32 can be inserted into the mesh 12 through opening 30. The tubular configuration of the mesh 12 can help to guide the applicator 32 toward one of the tips 16 of the sling 10 as the applicator 32 parts tissue growth that may have occurred in the mesh 12. The applicator 32, upon reaching the tip 16, can be used to push or aid in pulling the tip 16 to increase or lessen the tension in the sling 10 as discussed above.

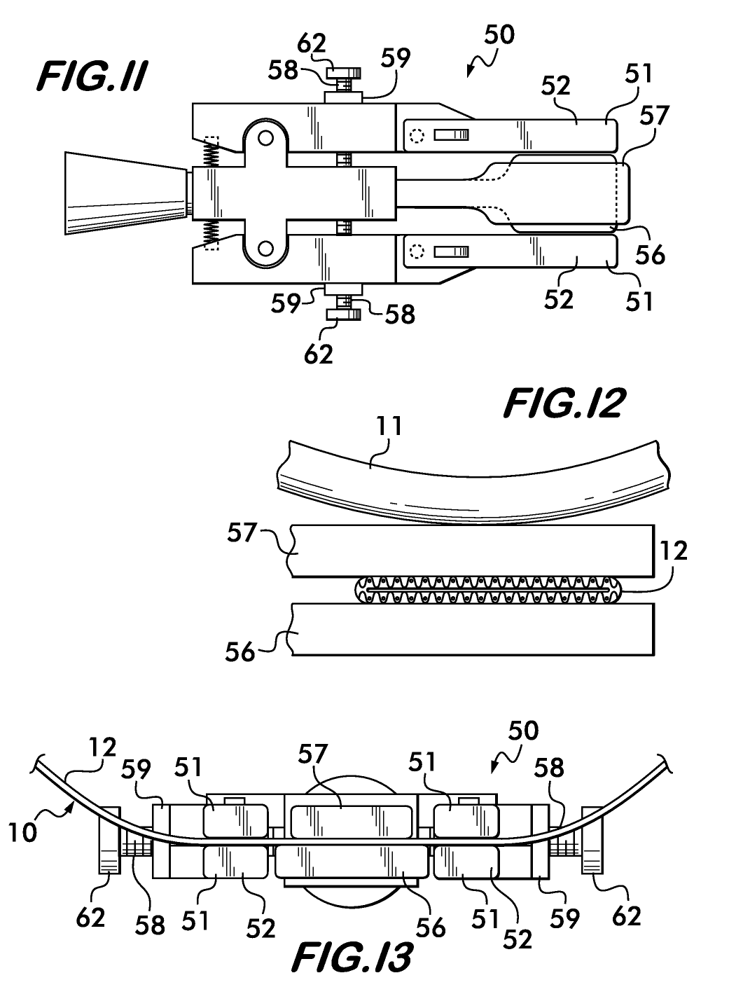

In an alternative embodiment, the tension in the sling 10 may also be adjusted on an intra- or post-operative basis without moving the tips 16; but rather, by applying an energy source to reduce or stretch the length of the mesh 12. Referring to FIGS. 10, 11, and 13 in certain embodiments, a gripping apparatus 50 may be used in combination with the energy source. The gripping apparatus 50 may include at least one grip 52, with certain embodiments, as illustrated FIG. 10, comprising a pair of grips 52 adapted to spread and contract the mesh sling. Each grip 52 includes a pair of handles 51 configured to securely grasp the mesh 12. In one non-limiting embodiment, for example, a pair of handles 51 can be rotatable about a pivot pin. The grip 52 may be further equipped with a spring element adapted to force the handles toward one another at one end and to facilitate the grip 52 in grasping the mesh 12. The grips 52 can grasp the mesh 12 on either or both sides of the portion of the mesh 12 located directly below the urethra 11, i.e., the central portion of the mesh 12, as shown in FIGS. 10 and 13.

The gripping apparatus 50 further includes provisions that permit the left and right-hand pairs of grips 52 to be moved toward and away from each other, and locked in position. For example, referring to FIGS. 10 and 11, the gripping apparatus 50 can include: a threaded member 58 passing between two grips 52. A threaded fitting fixed 59 to one of the grips engages the threaded member 58 as shown in FIG. 10 and secures the threaded member thereto. A second fitting 59 fixed to the other grip also engages the threaded member 58 and retains the other grip on the threaded member 58. In certain embodiments, the threaded member 58 is permitting to turn in relation to the grips 52. A knob 62 is disposed on one end of the threaded member 58. The knob facilitate turning of the threaded member 58 in relation to the grips 52 so as to facilitate the travel of the grips 52 along the longitudinal axis of the threaded member.

In certain embodiments, an energy source 56 can be moved into proximity or can be contained within the handle or body of the device with the central portion of the mesh 12, as shown in FIGS. 10-13. The energy source 56 can be any device suitable for altering the length of the central portion of mesh 12 between the handles in a controlled, focused manner, to induce shrinkage or stretching in the mesh 12. For example, the heat source 56 can be a heat gun (or otherwise a temperature changing source) or a light source (e.g. UV, IR, etc.) such as a surgical light wand without a heat sink, or any other suitable device capable of facilitating stretching or shrinking the material of the instant sling 10.

A protective shield 57 can also be positioned between the urethra 11 and the central portion of the mesh 12, as shown in FIGS. 11-13. The shield 58 can be any device that is capable of insulating the urethra from the energy emitted by the source (e.g. heat from heat source, light from a light source, etc). The shield 57 is, in certain embodiments, suitable for insertion between the mesh 12 and the urethra 11 or surrounding tissue.

In practice, the sling 10 can be shrunk in instances where the tension in the sling 10 needs to be increased, i.e. where the sling needs to be shortened for a better fit. In particular, gripping apparatus may be coupled to the sling as noted above and illustrated in FIGS. 10-13 and the knob 62 rotated once the grips 52 have grasped the mesh 12. Again, this causes the left-hand and right-hand pairs of grips 52 to move toward each other and the central portion of the mesh 12 to become slack. The energy source is then applied to facilitate shrinking the mesh. For example, in one non-limited embodiment, the central portion of the mesh 12 is then heated using a heat source. The heating can be ceased after the temperature of the central portion of the mesh 12 has increased sufficiently to facilitate shrinkage of the mesh 12, i.e. the mesh 12 can be heated for a predetermined time based on the known thermal characteristics of the mesh 12. The mesh 12 can be held in the grips 52 as it cools. The resulting shrinkage of the central portion of the mesh 12 causes the overall length of the sling 10 to decrease, which in turn causes the tension in the sling 10 to increase once the mesh 12 is released from the grips 52.

The sling 10 also can be stretched in instances where the tension in the sling 10 needs to be decreased. In particular, the knob 62 can be rotated once the grips 52 have grasped the mesh 12 as discussed above, so that the grips 52 move away from each other. This action causes the central portion of the mesh 12 to stretch. The energy source is then applied to facilitate stretching the mesh. For example, the central portion of the mesh 12 can then be heated using a heat source. The heating can be ceased after the temperature of the central portion of the mesh 12 has decreased sufficiently to facilitate permanent deformation of the central portion of the mesh 12 to its stretched length, i.e. the mesh 12 can be heated for a predetermined time based on the known thermal characteristics of the mesh 12. The mesh 12 can be held in the grips 52 as it cools, to prevent the central portion of the mesh 12 from shrinking or returning to its original size is it cools. The resulting lengthening of the central portion of the mesh 12 causes the overall length of the sling 10 to increase, which in turn causes the tension in the sling 10 to decrease once the mesh 12 is released from the grips 52.

In alternative embodiments, however, shrinking or stretching the sling 10 may be accomplished without use of a gripping apparatus 50. Rather, the length of the sling 10 may be altered by simply applying the energy source to a localized area of the sling. The amount of shrinking or stretching may be a function of the intensity of the energy source combined with known properties of the fibers used within the woven mesh and without otherwise having to manipulate the sling. To this end, shrinkage or stretching may be precisely controlled by regulating the amount of energy applied thereto by the energy source. These alternative methods can be performed in applications where the stretching or shrinkage characteristics of the mesh 12 are known and can be precisely controlled by regulating the amount of energy applied thereto by the energy source.

The above-described processes for shortening or lengthening the sling 10 is not necessarily limited to the sling of the instant invention. In alternative embodiment, it may also be performed on a conventional flat-mesh sling as discussed herein. Moreover, portions of the mesh 12 other than the central portion can be grasped, heated, and/or shortened in the alternative.

Specific details of the gripping apparatus 50 are presented for exemplary purposes only. The mesh 12 can be grasped, and stretched or contracted using similar or otherwise known means in the alternative.

The foregoing description is provided for the purpose of explanation and is not to be construed as limiting the invention. Although the invention has been described with reference to preferred embodiments or preferred methods, it is understood that the words which have been used herein are words of description and illustration, rather than words of limitation. Furthermore, although the invention has been described herein with reference to particular structure, methods, and embodiments, the invention is not intended to be limited to the particulars disclosed herein, as the invention extends to all structures, methods and uses that are within the scope of the appended claims. Those skilled in the relevant art, having the benefit of the teachings of this specification, can make numerous modifications to the invention as described herein, and changes may be made without departing from the scope and spirit of the invention as defined by the appended claims.

What is claimed is:

1. A urethral sling, comprising: a longitudinally extending tubular mesh having first and second ends.

2. The urethral sling of claim 1, wherein the tubular mesh comprises fibers woven into a tubular shape.

3. The urethral sling of claim 2, wherein the fibers are formed from a material selected from the groups consisting of a bioabsorbable material, a biocompatible material, a thermoplastic material, and combinations thereof.

4. The urethral sling of claim 2, wherein the fibers are formed from a material selected from the group consisting of polypropylene, polydioxanone and combinations thereof.

5. The urethral sling of claim 2, wherein the fibers are small gage fibers.

6. The urethral sling of claim 1, further comprising an opening in the mesh to permit an applicator to be inserted into the mesh.

7. The urethral sling of claim 1, wherein the mesh is collapsible into the substantially flat configuration.

8. The urethral sling of claim 1 further comprising a tip attached to at least one of said first and second ends of the mesh.

9. The urethral sling of claim 8 further comprising a first tip attached to the first end of the mesh; and a second tip attached to the second end of the mesh.

10. The urethral sling of claim 8, wherein the tip comprises a substantially circular body, and a conical end portion adjoining the body wherein the end of the mesh is attached to the body of the tip.

11. The urethral sling of claim 10, wherein the body and the conical end portion of each of the tip are hollow or semi-hollow so as to receive an end of the applicator.

12. The urethral sling of claim 8, wherein the tip is formed from a material selected from the groups consisting of a bioabsorbable material, a biocompatible material, a thermoplastic material, and combinations thereof.

13. The urethral sling of claim 8, wherein the tip is formed from a material selected from the group consisting of polypropylene, polydioxanone and combinations thereof.

14. The urethral sling of claim 1, further comprising a saddle fastened to or woven within the mesh.

15. The urethral sling of claim 14, wherein the saddle is approximately positioned at a length-wise midpoint of the mesh such that it contacts the urethra when positioned within the patient.

16. The urethral sling of claim 14, wherein the saddle is configured to support the urethra.

17. The urethral sling of claim 1, wherein the mesh is formed from a material that shrinks or stretches when heated.

18. A method for implanting or adjusting a urethral sling in a patient, the urethral sling comprising a tubular mesh, and a tip optionally attached to an end of the mesh, the method comprising:
i. inserting an applicator into the mesh via an opening formed in the mesh so that an end of the applicator engages an end of the sling; and
ii. pushing the end of the sling to a desired location within the patient using the applicator.

19. The method of claim 18, further comprising adjusting a portion of the urethral sling after pushing the end of the sling to a desired location within the patient using the applicator.

20. The method of claim 19, where adjusting a portion of the urethral sling after pushing the end of the sling to a desired location within the patient using the applicator comprises reinserting the applicator into the mesh via the opening formed in the mesh so that the end of the applicator engages the end of the sling, and pushing or pulling the end of the sling using the applicator.

21. A method for adjusting tension in a urethral sling, comprising;
i. grasping the sling in at least two positions;
ii. adjusting the length of the sling between the two positions;
iii. placing an energy source in proximity to a portion of the sling between the two positions; and
iv. altering the length of the sling using the energy source.

22. The method of claim 21, wherein the energy source is either a heat source or a light source.

23. The method of claim 21, wherein the length of sling between the two positions is adjusted by moving the two positions toward or away with respect to each other.

24. The method of claim 21, further comprising placing a thermally-insulating device between the mesh and a urethra supported by the urethral sling.

25. A method for adjusting tension in a urethral sling, comprising:
i. grasping the sling in at least two positions;
ii. adjusting the length of the sling between the two positions;
iii. placing an heating source in proximity to a portion of the sling between the two positions; and
iv. adjusting the length of the sling using the heat source.

26. The method of claim 25, wherein the heating source heats the sling to a temperature at or above which the mesh will permanently deform.

27. The method of claim 25, wherein the length of sling between the two positions is adjusted by moving the two positions toward or away with respect to each other.

28. The method of claim 25, further comprising placing a thermally-insulating device between the mesh and a urethra supported by the urethral sling.

29. A gripping apparatus for shrinking or stretch a urethral sling comprising:
a handle;
two or more gripping elements wherein the gripping elements are adapted to grip the urethral sling in a least two positions; and
and energy source.

30. The gripping apparatus of claim 29 further comprising a threaded element passing between the two or more gripping elements such that the threaded element controls movement of the gripping element toward and away from each other.

31. The gripping apparatus of claim 29 wherein the energy source is either a heat source or a light source.

32. The gripping apparatus of claim 29 wherein the energy source is positioned between two gripping elements.

33. The gripping apparatus of claim 29 further comprising a protective shield.

34. A method for adjusting tension in a urethral sling, comprising;
i. placing an energy source in proximity to a portion of the sling; and
ii. altering the length of the sling using the energy source.

35. The method of claim 34, wherein the energy source is either a heat source or a light source.

36. The method of claim 34, wherein the length of sling is altered based upon an amount of energy applied to the sling.

37. The method of claim 34, wherein the urethral sling is comprised of a woven mesh of formed from a material selected from the groups consisting of a bioabsorbable material, a biocompatible material, a thermoplastic material, and combinations thereof.

38. The method of claim 37, wherein the woven mesh is formed from a material selected from the group consisting of polypropylene, polydioxanone and combinations thereof.

39. The method of claim 37 wherein the length of the sling is altered based upon one or more properties of the one or more materials used to form the woven mesh and an amount of energy applied to the sling.
